(19) United States
(12) Patent Application Publication
(10) Pub. No.: US 2009/0317487 A1
(43) Pub. Date: Dec. 24, 2009

(54) KITS FOR PREVENTION AND TREATMENT OF RHINITIS

(76) Inventors: Mischelle Hall, Alpharetta, GA (US); Alan Roberts, Woodstock, GA (US); Matthew Heil, Sherman, CT (US)

Correspondence Address: SHERIDAN ROSS PC, 1560 BROADWAY, SUITE 1200, DENVER, CO, 80202, US

(21) Appl. No.: 12/549,213

(22) Filed: Aug. 27, 2009

Related U.S. Application Data
(63) Continuation of application No. 11/622,998, filed on Jan. 12, 2007.
(60) Provisional application No. 60/825,845, filed on Sep. 15, 2006.

(51) Int. Cl. A61K 33/30; A61P 31/16; B65D 83/04

(52) U.S. Cl. 424/641; 206/531

(57) ABSTRACT

Kits providing a combination of one or more pharmaceutical information comprising one or more agent(s) for the treatment or alleviation of symptoms commonly associated with a cold and an immunonutritional composition comprising immunonutritional agent and methods of using these kits are described. The kits provide both the pharmaceutical agent(s) and the immunonutritional agent in a convenient form for administration. The kit typically includes instruction for coordinating the administration of the pharmaceutical formulation with the administration of the immunonutritional composition. The preferred immunonutritional agents are compounds that contain a pharmaceutically acceptable form of zinc, such as zinc acetate, zinc gluconate, zinc gluconate glycine, and zinc sulfate. Preferably the kit contains multiple dosage forms containing the immunonutritional composition. In the most preferred embodiment, the immunonutritional composition is in the form of a lozenge. Suitable pharmaceutical agents include but are not limited to antihistamines, decongestants, anticholinergics, antitussives, analgesics, mucolytics, expectorants, and combinations thereof. The pharmaceutical formulations may be in any suitable dosage form, including forms which provide controlled release of the pharmaceutical agent, including immediate, sustained, modified, delayed or pulsed release pharmacokinetic mechanism or a combination thereof. The combined treatment requires administration of both the pharmaceutical formulation(s) for the treatment of symptoms commonly associated with a cold and the administration of the immunonutritional composition, which supplies nutritional support for the patient's innate immune response to the presence of infectious organisms.

### CROSS-REFERENCE TO RELATED APPLICATIONS

The present application is a continuation of U.S. patent application Ser. No. 11/622,998, filed on Jan. 12, 2007, which in turn claimed priority to U.S. Ser. No. 60/825,845, filed in the United States Patent and Trademark Office on Sep. 15, 2006.

### FIELD OF THE INVENTION

The invention is generally in the field of kits for the prevention and/or treatment of rhinitis and its symptoms.

### BACKGROUND

A wide variety of bacterial, viral and external irritants can result in upper respiratory inflammation. Infectious rhinitis, which is usually referred to as the “common cold”, is the most prevalent form of rhinitis. Colds are caused by viruses, which are a distinct class of biologic organisms from bacteria. A popular misconception behind the common cold is the confusion between viruses and bacteria as etiological agents. Because of this misconception many patients, or their guardians, incorrectly believe that use of an antibiotic will assist in limiting the course and extent of the cold and assist in healing, which has been shown to be untrue.

Among the viruses known to cause rhinitis are more than 100 known variants of rhinovirus alone. The facility with which these viruses are spread, aided by their high virulence (it is estimated that only 1-30 viral particles are needed for successful infection), emphasizes the need for treatments that address symptoms as well as the causes of infectious rhinitis.

Another form of rhinitis is allergic rhinitis, which results from an IgE-based response triggering the release of inflammatory chemokines in response to a sensitizing allergen. Other form of rhinitis, such as vasomotor rhinitis, result from entirely different mechanisms involving selective neurologic interaction with the smooth musculature of the circulatory system.

Infection by a viral pathogen capable of producing rhinitis results in a rapid response by the immune system characterized by the release of several proinflammatory cytokines such as the leukotrienes, the mass migration of neutrophils and macrophages, infiltration by T-cell lymphocytes and alterations in the permeability of the endothelial lining of the local blood vessels.

Because there are a multiplicity of viruses that can cause infectious rhinitis, treatment presents its own special needs. The current therapeutic paradigm has emphasized utilization of pharmacologic agents to address a patient's symptoms. This treatment is frequently combined with other pharmacologic agents, such as antibiotics, which are intended to target the cause of the infection driving the symptoms. However, it is a well understood principal of medicine that antibiotics are active only against bacteria. The action of many antibiotics relies upon interference with the proper construction of the bacterial cell wall, leading to incomplete and non-viable reproduction of the bacterium. Viruses, the most common cause of infectious rhinitis, do not even have cell walls and are radically different from bacteria rendering them unaffected by antibiotics. Additionally, the use of antibiotics in the treatment of a cold can increase the severity of a cold by unintentionally eliminating many beneficial bacteria in the body, thereby creating an environment more hospitable to viral overgrowth.

Despite these issues, the paradigm for treating infectious rhinitis remains sound. Relief of the patient's immediate symptoms and trying to address the underlying cause of those symptoms are goals common to the treatment of virtually all diseases. There is a need, however, for a therapeutic regimen which can address the symptomology of infectious rhinitis and also assist in the clearance of the infectious viruses from the body. While antiviral agents do exist, their side effects, cost and limited effectiveness make them poor choices on a therapeutic risk/benefit basis. There exists a need for compositions to treat the cause of the infection underlying a cold as well as its symptoms by utilizing conventional pharmacologic treatment for cold symptoms combined with immunonutrition that assists the patient's own immune response, which in turn can directly target and destroy viral pathogens.

Several nutritional studies have demonstrated a link between nutrition and proper functioning of the immune system. For example, immune cells taken from nutritionally deficient individuals have been shown to have a reduced capacity to produce necessary cytokines which are a major factor driving the immune response to a variety of infectious agents (Savendahl, L and Underwood, L E, J. Clin. Endocrinol. Metab., 82:117-80 (1997). Utilization of immunonutritional supplementation has been an important part of mainstream medicine for several years. A study completed in 1998 surveying the use of non-pharmacologic treatments by conventional physicians, as well as those specifically treating patients suffering from asthma, showed diet and nutrition to be the most often cited complementary therapy used in conjunction with drug therapy (Davis P A, et al., J. Investig. Allergol. Clin. Immunol. 8:73-7 (1998).

Studies of nutrition have specifically identified key nutritional elements, the lack of which can have a profound negative impact on the immune system. For example, as reported by Cunningham-Rundles et al., a lack of macro- as well as micronutrients such as, zinc, iron and the anti-oxidant vitamins, can lead to significant immunodeficiency, particularly in children (Cunningham-Rundles, S and McNeeley, D F. J. Allergy Clin. Immunol., 115:1119-1128 (2005); Keusch G T, J. Nutr., 133:336 S-40S (2003)). Schneider et al. have shown hospital nutrition programs that do not address the needs of immunonutrition can increase the likelihood of nosocomial infections (Schneider, et al., Br. J. Nutr., 92:205-11 (2004)).

In addition to lowering the efficacy of the immune response, it has also been shown that infections are more frequent in the immunonutritionally deficient and there is a greater chance that these infections, once contracted, will become chronic (Cunningham-Rundles, S and McNeeley, D F, J. Allergy Clin. Immunol., 115:1119-1128 (2005)). A significant part of the mechanism behind this increased incidence and chronicity of infections lies with defects found in the cellular immune response. When key nutritional elements necessary for the immune system are missing from the diet over time, the functioning of phagocytic cells, such as macrophages and neutrophils, is decreased (Savino W., Eur. J. Clin. Nutr., 56(suppl 3):S46_{—}9 (2002); Najera, et al., Clin. Exp. Immunol., 126:461-5 (2001)). Proper functioning of these cells is essential for clearance of host cells that harbor pathogenic viruses. The overall result could be altered microbial colonization of mucosal surfaces in the sinuses and oropharynx as well as an impaired host response to new pathogens. Such alterations can increase the likelihood of successful infection with a cold virus. The importance of these effects is particularly highlighted by the fact that very small doses of cold virus are sufficient to produce infection. 1-30 viral particles have been shown to be sufficient to produce infection in test subjects (Hendley, J. O. and J. M. Gwaltney, Jr., Epidemiologic Review, 10:243-258 (1988); Douglas, R. G., J. Ann. Otol. Rhinol. Laryngol., 79:563-571 (1970).

The use of nutritional supplementation with zinc has been shown to have a clear effect on the duration and extent of symptoms associated with infectious rhinitis. Studies conducted in 1996 showed the zinc supplementation has a significant effect on the severity and duration of infectious rhinitis (Mossad, S B, Ann. Intern. Med., 125(2):81-8 (1996)). Another study demonstrated that the administration of zinc acetate supplements to patients in the initial stages of infectious rhinitis resulted in a reduced duration of infection (Prasad, A S. Ann. Intern. Med., 133(4):245-52 (2000)). Tests with zinc have also shown an ability to suppress inflammation in the throat associated with infectious rhinitis (Novick, S G, Med. Hypotheses, 49(4):347-57 (1997)).

Zinc is required for a number of other immune functions, including T-lymphocyte activity. Zinc deficiency results in thymic involution, depressed delayed hypersensitivity, decreased peripheral T-lymphocyte count, decreased T-cell response to proliferative signals (e.g. PHA), decreased cytotoxic T-lymphocyte activity, depressed T helper lymphocyte function, depressed natural killer cell activity, depressed macrophage function (phagocytosis), depressed neutrophil functions (respiratory burst, chemotaxis) and depressed antibody production. Zinc supplementation can restore impaired immune function in those with zinc deficiency.

The mechanism underlying the immune effects of zinc is not fully understood. Some of these effects may be accounted for by zinc's membrane-stabilization effect. This could affect signaling processes involved in cell-mediated mediated immunity. Zinc is known to be involved in such signaling processes. Zinc may also influence gene expression by structural stabilization of different immunological transcription factors. Zinc ions can induce blast formation of human peripheral blood monocytes (PBMCs). In PBMCs, zinc induces cytokines, including interleukin (IL)-1, IL-6 and tumor necrosis factor (TNF)-alpha. Cytokine induction by zinc is caused by a direct interaction of zinc with monocytes. The stimulation of T-lymphocytes by zinc appears to occur via monocyte released IL-1 and cell-cell contact. High zinc concentrations inhibit T-lymphocyte proliferation by blocking the IL-1 type 1 receptor-associated kinase. T-lymphocyte activation appears to be delicately regulated by zinc concentrations.

Zinc may also have secondary antioxidant activity. Zinc does not have redox activity under physiological conditions. Zinc may influence membrane structure by its ability to stabilize thiol groups and phospholipids. It may also occupy sites that might otherwise contain redox active metals such as iron. These effects may protect membranes against oxidative damage. Zinc also may be in the form of copper/zinc-superoxide dismutase (Cu/Zn-SOD). Zinc may also have antioxidant activity via its association with the copper-binding protein metallothionein.

The immune system is adversely affected by even moderate degrees of zinc deficiency. Severe zinc deficiency can dangerously depress immune function (Shankar A H and Prasad A S, Am. J. Clin. Nutr., 68:447 S-463S (1998)). Zinc is required for the development and activation of T-helper/suppressor lymphocytes, a kind of white blood cell that controls the immune response (Institute of Medicine, Food and Nutrition Board, Directory Reference Intakes for Vitamin A, Vitamin K, Arsenic, Boron, Chromium, Copper, Iodine, Iron, Manganese, Molybdenum, Nickel, Silicon, Vanadium, and Zinc, National Academy Press, Washington, D.C. (2001); Beck F W, et al., Am. J. Physiol., 272:E1002-1007 (1997)). When zinc supplements are given to individuals with low zinc levels, the numbers of T-cell lymphocytes circulating in the blood increase and the ability of lymphocytes to fight infection improves. Zinc supplementation studies, using from 4 mg/day to 40 mg/day, have successfully used zinc delivered in a variety of forms including zinc acetate, zinc gluconate, or zinc sulfate.

Many commercial formulations that contain a zinc ion source contain one or more pharmaceutically acceptable excipients which can complex, or sequester, metallic ions such as zinc to form stable, neutral or negatively charged complexes. This complexation results in the deactivation of zinc and a decrease in the effectiveness of the formulation. Examples of sequestering agents include organic acids such as citric acid, sweeteners such as saccharin, sorbitol, mannitol, and aspartame, and flavoring agents. For Example, U.S. Pat. No. 6,793,942 to Gelber et al. describes medicinal compositions containing a pain reliever, an anti-inflammatory pharmaceutical and a neutriceutical, such as zinc, in a pharmaceutically acceptable base. The compositions contain organic acids, such as ascorbic acid, and other excipients or active ingredients which can complex zinc.

In a 1987 study, Farr et al. showed that formulations containing sufficient amounts of citric acid, a known strong zinc chelator, to eliminate the taste of zinc gluconate showed no reduction in duration of common colds. The first stability constant of citric acid for zinc ions is generally accepted to be log K.sub.1=4.5. In oral use in lozenge form, zinc gluconate rapidly ionizes. It is known in the art that if this ionization occurs in the presence of sufficient amounts of a chelator having a high stability constant for zinc ions, such as equimolar or extramolar citric acid, a new, vastly stronger equilibrium replaces the weak association with gluconate which can eliminate the availability of metallic ions at pH 7.4. In the Farr experiment such zinc citrate equilibrium resulted in neutral and negatively charged compounds having no bioavailability at normal oral tissue pH according to Berthon, May and Williams, Journal of the Chemical Society, Dalton, 1433-1438 (1978). In the case of lozenges containing zinc gluconate with extramolar citric acid, soluble zinc citrate complexes where shown to be tasteless and were proposed to be sufficiently biologically available to be effective in reducing duration of common colds. However, with addition of extramolar citric acid, there occurs in saliva such powerful binding of zinc ions that neutral or negatively charged zinc species predominate. A near complete loss of positively charged Zn.sup.2+ ions occurs in saliva at pH 4.3 and a complete loss of Zn.sup.2+ ions occurs in oral tissues at pH 7.4. No localized activity occurs, and no reduction in common cold duration occurs from zinc tightly bound by citrate.

It is an object of the invention to provide improved methods for the prevention and/or treatment of rhinitis.

It is a further object of the invention to provide kits for the prevention and/or treatment of rhinitis.

### SUMMARY OF THE INVENTION

Kits providing a combination of one or more pharmaceutical formulations containing one or more agent(s) for the treatment or alleviation of symptoms commonly associated with a cold and an immunonutritional composition containing an immunonutritional agent and methods of using these kits are described. The kits provide both the pharmaceutical agent(s) and the immunonutritional agent in a convenient from for administration. The kit typically includes instructions for coordinating the administration of the pharmaceutical formulation with the administration of the immunonutritional composition. The preferred immunonutritional agents are compounds that contain a pharmaceutically acceptable form of zinc including, but not limited to, zinc acetate, zinc gluconate and zinc sulfate. Preferably the kit contains multiple dosage forms containing the immunonutritional composition. In one embodiment, the immunonutritional composition is in the form of a lozenge and the pharmaceutical formulation is in the form of a solution, suspension, tablet, capsule, sustained release capsule, or chewable tablet. In another embodiment, the immunonutritional composition and the pharmaceutical composition are liquids, such as solutions or suspensions, which can be administered orally or nasally. The compositions can also be formulated as gels. Suitable pharmaceutical agents include, but are not limited to antihistamines, decongestants, anticholinergics (including anticholinergic antisecretory agents), antitussives (including narcotic antitussives such as codeine, hydrocodone, hydromorphone, pholcodeine, ethylmorphine, methadone and dihydrocodiene), analgesics, mucolytics, expectorants, and combinations thereof. The pharmaceutical formulations may be in any suitable dosage form, including forms which providing controlled release of the pharmaceutical agent, including immediate, sustained, modified, delayed or pulsed release formulations or a combination thereof. The combined treatment requires administration of both the pharmaceutical formulations(s) for the treatment of symptoms commonly associated with a cold and the administration of the immunonutritional composition, which supplies nutritional support for the patient's innate immune response to the presence of infectious organisms.

In one embodiment, the kit contains zinc acetate lozenges and an oral solution containing guaifenesin, carbetapentane tannate, and phenylephrine tannate. In another embodiment, the kit contains zinc acetate lozenges and an oral solution containing hydrocodone bitartrate, guaifenesin, and phenylephrine hydrochloride. In still another embodiment, the kit contains zinc acetate lozenges and chewable tablets containing chlorpheniramine maleate, phenylephrine hydrochloride, and methscopolamine nitrate. In yet another embodiment, the kit contains zinc acetate lozenges and an oral suspension containing carbetapentane tannate and phenylephrine tannate. In still another embodiment, the kit contains zinc acetate lozenges and capsules containing pseudoephedrine hydrochloride and chlorpheniramine maleate.

### DETAILED DESCRIPTION OF THE INVENTION

### I. Definitions

The term “immunonutritional” refers to those compounds, materials, compositions, and/or dosage forms which, whining the scope of sound nutritional or medical judgment, are suitable for use as nutrients including, but not limited to, zinc, minerals and vitamins and combinations thereof, that have demonstrated utility in the biological functioning of the immune system.

The terms “pharmacologic agent” and “pharmaceutical agent” are used herein interchangeably to refer to agents intended for use in the diagnosis, mitigation, treatment, cure, or prevention of disease in man or in other animals.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

An “antibiotic” means a compound which kills or inhibits infection, proliferation or the effects of a single celled organism, such as bacteria or yeast.

The term “adjunctive administration”, as used herein, means simultaneous administration of the compounds, in the same dosage form, simultaneous administration in separate dosage forms, and/or separate administration of the compounds.

A “cold” refers specifically to acute rhinitis resulting from infection with any of a number of viral agents including but not limited to members of the rhinovirus and coronavirus families.

“Effective amount” or “therapeutically effective amount” means the amount needed for the desired therapeutic effect and includes any additional amount or overage of active ingredient deemed necessary in the formulation to provide the desired amount upon administration.

“Immediate Release” or “IR” means the therapeutic pharmaceutical composition is provided in a formulation allowing the active agent to begin acting in a therapeutic manner substantially as soon as the agent becomes available in the body and/or bloodstream of the patient.

A “delayed release dosage form” is one that releases a drug (or drugs) at a time other than promptly after administration.

An “extended release dosage form” is one that allows at least a twofold reduction in dosing frequency as compared to that drug presented as a conventional dosage form (e.g. as a solution or prompt drug-releasing, conventional solid dosage form).

A “modified release dosage form” is one for which the drug release characteristics, time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

“Pulsed release” refers to an initial release of drug, followed by a period of substantially no release, followed by one or more additional releases of drug separated by a period of substantially no release. This does not mean that there are no blood levels of drugs between periods of release.

“Sustained release” or “SR” means the therapeutic pharmaceutical composition is provided in a formulation such that the composition provides an initial therapeutic effect and also an ongoing or additional release of the therapeutic pharmaceutical composition or therapeutic effect over a desired period of time.

“Substantially no liver toxicity” means that a patient ingesting a therapeutic pharmaceutical composition does not experience a substantial increase in liver enzyme production associated with administration of the composition.

### II. Kits

The kits contain a combination of one or more pharmaceutical agent(s) for the treatment or alleviation of symptoms commonly associated with a cold and one or more dosage forms containing an immunonutritional agent, preferably zinc.

The kits are packaged to provide both the pharmaceutical agent(s) and the immunonutritional agent in a convenient from for administration. In one embodiment, the kit contains multiple solid oral dosage forms, such as tablets, chewable tablets, or capsules containing the active ingredient(s) in the same or different dosages. In another embodiment, the pharmaceutical formulation and/or the immunonutritional composition is in the form of a liquid, such as a solution or suspension, typically stored in one or more bottles or vials. This design provides a method to conveniently administer the pharmacologic agent(s), for example, in increasing dosages of the pharmaceutical agent, such as, beginning at 25 mg, gradually increasing to 50 mg, 75 mg, 100 mg, 200 mg, 400 mg, 500 mg, over a period ranging from three days up to 16 weeks. Alternatively, the design may be used to administer decreasing dosages of the pharmaceutical agent.

The packaging material may be a box, bottle, blister package, tray, or card. The kit may contain bottles for the storage of liquids. The kit typically includes instructions for coordinating the administration of the pharmaceutical agent with the administration of the immunonutritional agent. For example, the pharmaceutical agent may be administered at certain times during the day in order to control symptoms associated with infectious rhinitis, while the immunonutritional agent may be administered at the same or different times of the day to provide nutritional supplementation for support of the immune system during the treatment period. If the kit contains different dosage of the pharmaceutical formulation, the instructions indicate which dose should be administered and when the dose should be administered. For example, a first dose on day one, a second higher dose on day two, a third higher dose on day three, and so, until a maintenance dose is reached. Alternatively, the dose unit pack may contain multiple formulations designed to give different doses of the pharmaceutical agent, or to provide different drug combinations, that can be taken at different times, e.g. on different days or different times of the day.

A. Immunonutritional Composition

The immunonutritional composition provides nutritional support for the patient's innate immune response to the presence of infectious organisms. Immunonutritional agents, such as zinc and other minerals and vitamins, are well known in the art and are available commercially from numerous sources well known to those skilled in the art.

In a preferred embodiment, the immunonutritional composition delivers a therapeutically effective amount of zinc in the range of 2 mg/day to 60 mg/day. In a preferred embodiment, the immunonutritional composition contains zinc in an appropriate chemical form including, but not limited to, zinc acetate, zinc gluconate, zinc gluconate glycine, and zinc sulfate.

Immunonutritional compositions containing zinc typically exclude organic acids, such as citric acid and ascorbic acid, and other agents which are capable of complexing or sequestering zinc ions, which have been shown to reduce the bioavailability of zinc in vivo. The compositions may also include excipients, such as flavoring agents and others excipients to enhance palatability and to provide consistency in the manufacturing process. The kit typically contains multiple lozenges or tablets comprising the immunonutritional composition, which are designed to be used by the patient every 2 to 3 hours. A typical daily dose is up to 40 mg of elemental zinc in one day.

In a preferred embodiment, the immunonutritional composition contains a zinc-containing compound and contains at least 2 mg and no more than 20 mg of elemental zinc. In yet a more preferred embodiment, the immunonutritional composition contains from 5 mg to 15 mg of elemental zinc. In a most preferred embodiment the immunonutritional composition contains 12 mg of elemental zinc. Optimal dosage is adjusted according to the age of the patient, providing 2 mg/day to 10 mg/day supplementation for infants from birth to 3 years of age; 10 mg/day for children 4 to 12 years of age and 10 mg/day to 15 mg/day for children over the age of 12 and adults. The composition can be taken up to 10 times a day by adults for a maximum dosage of 140 mg of zinc.

B. Pharmaceutical Agents for Treating Symptoms of Rhinitis

The kit also includes formulations containing one or more pharmaceutical agents suitable for the treatment of symptoms associated with a cold for adjunctive administration with the immunonutritional compositions. These pharmaceutical agents include, but are not limited to, antihistamines, decongestants, anticholinergics, antitussives, analgesics, antipyretics, mucolytics and/or expectorants, alone or in combination with each other. The symptoms these pharmaceutical agents may alleviate include, but are not limited to, rhinorrhea, nasal inflammation, pharyngeal inflammation, sore throat, muscular aches, fever, sneezing, productive or unproductive cough, excess mucus production in the upper respiratory system, and watery eyes. Different combinations of the pharmaceutical agents may be used in the pharmaceutical formulations. For example, an antihistamine can be combined with a decongestant and an anticholinergic. Alternatively an antihistamine can be formulated alone or combined with a therapeutic dose of a decongestant.

Suitable antihistamines include, but are not limited to, chlorpheniramine, bromopheniramine, diphenhydramine, doxylamine, pheniramine, promethazine, clemastine, cyproheptadine, hydroxyzine, meclizine, and combinations thereof.

Suitable decongestants include, but are not limited to, phenylephedrine, pseudoephedrine, anticholinergics (including anticholinergic antisecretory agents), methscopolomine, hyocyamine, and combinations thereof.

Suitable antitussives include, but are not limited to, dextromethorphan, guaifenesin, codeine, hydrocodone, benzonatate, hydromorphone, pholcodeine, ethylmorphine, methadone and dihydrocodiene, and combinations thereof.

Examples of pharmaceutical agents suitable for the treatment of symptoms associated with a cold include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bennoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxaxzolam, clozapine, codeine, corticosterone, cortisone, cyclobenzaprine, cyproheptadine, demexiptiline, desipramine, desomorphine, dexamethasone, dexanabinol. dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, kitaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metazalone, methadone, methadone, methamphetamine, methocarbamol, methyldopa, methylphenidate, methylsalicylate, methysergid(e), metoclopramide, mianserin, mifepristone, methscopolamine, minaprine, mirtazapine, moclobemide, modafinil (an anti-narcoleptic), molinedone, morphine, morphine hydrochloride, nabumetone, nadolol, naproxen, naratriptan, nefazondeon, neurotin, nomifensine, nortriptyline, olanzapine, olsalazine, ondansetron, opipramol, orphenadrine, oxaflozane, oxaprazine, oxazepam, oxitriptan, oxycodone, oxymorphone, pancrelipase, parecoxib, paroxetine, pemoline, pentazoncine, pepsin, perphenazine, phenacetin, phendimetrazine, phenmetrazine, phenylbutazone, phenyloin, phosphatidylserine, pimozide, pirlindole, piroxicam, pizotifen, pizotyline, pramipexole, prednisolone, prednisone, pregabaline, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

i. Other Pharmaceutical Agents

Optionally, the pharmaceutical formulation contains additional pharmaceutical agents, other than the agents for the treatment of symptoms associated with a cold. Such additional pharmaceutical agents include, but are not limited to, anti-inflammatory drugs, antidepressants, antiepileptics, antimigraine drugs, antimuscarinics, anxiolytics, sedatives, hypnotics, antipsychotics, bronchodilators, anti asthma drugs, cardiovascular drugs, corticosteroids, dopaminergic, electrolytes, gastro-intestinal drugs, muscle relaxants, parasympathomimetics, stimulants, anorectics and anti-narcoleptics. This list of drugs includes, but is not limited to, chlorpheniramine, methascopolamine, scopolamine, phenylephrine, pseudoephedrine, hydrocodone, guaifenesin, dexchlorpheniramine, potassium guaiacolsulfonate, acetaminophen, aspirin, and other salicylates such as salicylamide and sodium salicylate, codeine, brompheniramine, buclizine, cinnarizine, clemastine, cyclizine, cyproheptadine, diphenhydramine, diphenylpyraline, doxylamine, meclozine, pheniramine, promethazine, triprolidine, ipratropium, tiotropium, aceprometazine, terfenadine, azelastine, loratadine, cetirizine, astemizole, carbetapentane, theophylline, ephedrine, dihydrocodeine, hydrocodone, hydromorphone, caramiphen, dextromethorphan, homatropine, pyrilamine, and caffeine.

The one or more pharmaceutical agents can exist as the free acid or base or as a pharmaceutically acceptable salt thereof. As used herein, “pharmaceutically acceptable salts” refer to derivatives of the compounds listed above, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric sulfamic, phosphoric, and nitric acids; and the salts prepared from organic acids such as aceteic, propionic, succinic, glycolic, stearic, lactic, malic, tartaric, citric, ascorbic, pamoic, maleic, hydroxymaleic, phenylacetic, glutamic, benzoic, salicylic, sulfanile, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate based or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts; Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

ii. Excipients

Pharmaceutical formulations are prepared using pharmaceutically acceptable “carriers” composed of materials that are considered safe and effective and may be administered to an individual without causing undesirable biological side effects or unwanted interactions. The term “carrier” refers to all components present in the pharmaceutical formulation other than the active ingredient or active ingredients. For solid oral dosage formulations, the term “carrier” includes, but is not limited to, diluents, binders, lubricants, disintegrators, fillers, preservatives, and coating compositions. The term “carrier” also includes all components of the coating composition, which may include plasticizers, pigments, colorants, stabilizing agents, and glidants. For liquid oral dosage forms, the term “carrier” includes, but is not limited to, preservatives, stabilizing agents, flavoring agents, sweeteners, dyes and colorants, solvents, viscosity modifiers, and pH modifying agents. The concentration of the one or more excipients is dependent on the formulation and can be readily determined by one of ordinary skill in the art. The Handbook of Pharmaceutical Excipients (5.sup.th Ed., Edited by Rowe, Shesky, and Owen) provides general ranges for a variety of excipients.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT.®. (Roth Pharma, Westerstadt, Germany), zein, shellac, and polysaccharides. The coating material may contain conventional excipients, such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Suitable preservatives include, but are not limited to, parabens, such as methylparaben and propylparaben.

Diluents, also referred to as “fillers”, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose (also known as hypromellose), hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric o nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER.®. 401, stearoyl monoisopropanolamide, and polyoxyethylene hydrogenated tallow amide. Examples of amphoteric surfactants include sodium N-dodecyl-.beta.-alanine, lauryl sulfobetaine.

If desired, the tablets, beads, granules or particles may also contain minor amount of nontoxic auxiliary substances such as wetting or emulsifying agents, dyes, pH buffering agents, and preservatives.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The preferred coating weights for particular coating materials may be readily determined by those skilled in the art by evaluating individual release profiles of tablets, beads and granules prepared with different quantities of various coating materials. It is the combination of materials, method and form of application that produce the desired release characteristics, which one can determine only from the clinical studies.

The coating composition may include conventional additives including, but not limited to, plasticizers, pigments, colorants, stabilizing agents, glidants, and combinations thereof. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as silicone (e.g., simethieone), may also be added to the coating composition.

Formulations may include additional excipients that can enhance the rate and extent of oral absorption of the pharmaceutical agent and/or the immunonutritional agent. Preferably, the formulation includes one or more absorption enhancers that increase the rate of absorption of the pharmaceutical agent and/or the immunonutritional agent across the buccal or intestinal mucosa, as compared to the same formulation in the absence of the absorption enhancer(s). Suitable absorption enhancers include, but are not limited to, surfactants, such as anionic and non-ionic surfactants; phospholipids, fatty acids, such as capric acid, and salts thereof; fatty acid glycerides; bile acids, such as cholic acid and deoxycholic acid; amino acids; mixed micelles; oil-in-water emulsions; chelating agents, such as EDTA and EGTA; glycyrrhizic acid; cyclodextrins, such hydroxypropyl-beta-cyclodextrin; polysaccharides, such as chitosans; liposaccharides; and ammonium glycerizinate.

iii. Dosage Forms

The pharmaceutical composition may be in any suitable form, including liquid and solid dosage forms. Examples of liquid dosage forms include, but are not limited to, oral solutions and suspensions and nasal sprays. Examples of solid and semi-solid dosage forms include, but are not limited to, tablets, chewable tablets, and capsules, films for lingual and/or buccal administration, and gels. Formulations with different drug release mechanisms could be combined in a final dosage form including single or multiple units. Examples of multiple units include multilayer tablets, capsules containing tablets, beads, granules, etc. in a solid or liquid form.

The immunonutritional composition can be formulated in a number of ways, such as in the form of a liquid or solid dosage form, for example, as solutions, suspensions, capsules, tablets, films, or delivery vehicles formulated for lingual and/or buccal administration.

Buccal administration can be achieved for periods of 30 minutes to 4 hours using formulations containing the active ingredient in combination with a bioerodible polymeric carrier such as described U.S. Pat. No. 6,221,329 to Place. Additional bioadhesive polymers include carbomer, partially hydrolyzed polyvinyl alcohol, polyethyleneoxide, polyacetate, hydroxypropylmethylcellulose (also known as hypromellose), and hydroxymethylcellulose. For oral delivery, the most preferred dosage form is a lozenge. The lozenge dissolves in a patient's mouth over a suitable period of time, preferably from 5 minutes to 30 minutes following administration. The immunonutritional composition is typically formulated for intermittent use during the day in concert with the pharmaceutical agent(s) for the treatment or alleviation of cold symptoms.

Immediate Release Formulations

Typical immediate release formulations include compressed tablets, gels, films, coatings, liquids and particles that can be encapsulated, for example, in a gelatin capsule. Many methods for preparing coatings, covering or incorporating drugs, are known.

The immediate release dosage unit of the dosage form, i.e., tablet, a plurality of drug-containing beads, granules or particles, or an outer layer of a coated core dosage form, contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads). A preferred method for preparing immediate release tablets (e.g., as incorporated into a capsule) is by compressing a drug-containing blend, e.g., blend of granules, prepared using a direct blend, wet-granulation or dry-granulation process. Immediate release tablets may also be molded rather than compressed, starting with a moist material containing a suitable water-soluble lubricant. However, preferred tablets described herein are manufactured using compression rather than molding. A preferred method for forming immediate release drug-containing blend is to mix drug particles directly with one or more excipients such as diluents (or fillers), binders, disintegrants, lubricants, glidants, colorants or the like. As an alternative to direct blending, a drug-containing blend may be prepared by using a wet-granulation or dry-granulation process. Beads containing the active agent may also be prepared by any one of a number of conventional techniques, typically starting from a fluid dispersion. For example, a typical method for preparing drug-containing beads involves blending the active agent with conventional pharmaceutical excipients such as microcrystalline cellulose, starch, polyvinylpyrrolidone, methylcellulose, talc, metallic stearates, silicone dioxide, or the like. The admixture is used to coat a bead core such as a sugar sphere (or so-called “non-pareil”) having a size of approximately 20 to 60 mesh.

An alternative procedure for preparing drug beads is by blending the drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating the bead to form immediate release beads.

Extended or Sustained Release Dosage Forms

Extended release formulations are generally prepared as diffusion or osmotic systems, for example, as described in “Remington—The science and practice of pharmacy”, 20th Ed., Lippincott (Williams & Wilkins, Baltimore, Md., 2000). A diffusion system typically consists of one of two types of devices, reservoir and matrix, and is well-known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose (also known as hypromellose), sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

An immediate release portion can be added to the extended release system by means of either applying an immediate release layer on top of the extended release core using coating or compression process or in a multiple unit System such as a capsule containing extended and immediate release beads.

Extended release tablets containing hydrophilic polymers are prepared by techniques commonly known in the art such as direct compression, wet granulation, or dry granulation processes. Their formulations usually pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

Delayed Release Dosage Forms

Delayed release dosage formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically polymers, as will be appreciated by those skilled in the art, become soluble in the higher pH environment of the lower gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT.®. (Rohm Pharma; Westerstadt, Germany), including EUDRAGIT.®.L30D-55 and L100-55 (soluble at pH 5.5 and above), EUDRAGIT.®. L-100 (soluble at pH 6.0 and above, EUDRAGIT.®. S (soluble at PH 7.0 and above, as a result of a higher degree of esterification), and EUDRAGITs.®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylase and guar gum; zein and shellac. Combinations of different coating materials may also be used. Multi-layer coatings using different polymers may also be applied.

The preferred coating weights for particular coating materials may be readily determined by those skilled in the art by evaluating individual release profiles or tablets, beads and granules prepared with different quantities of various coating materials. It is the combination of materials, method and form of application that produce desired release characteristics, which one can determine only from the clinical studies.

Alternatively, a delayed release tablet may be formulated by dispersing the drug within a matrix of a suitable material such as a hydrophilic polymer or a fatty compound. The hydrophilic polymers may be comprised of polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

Pulsed Release Forms

A pulsed release dosage form is one that mimics a multiple dosing profile without repeated dosing and typically allows at least a twofold reduction in dosing frequency as compared to the drug presented as a conventional dosage form (e.g. as a solution or prompt drug-releasing, conventional solid dosage form). A pulsed release profile is characterized by a time period of no release (lag time) or reduced release followed by rapid drug release.

Each dosage form contains a therapeutically effective amount of active agent. In one embodiment of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 30 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

For dosage forms mimicking a three times daily dosing profile, approximately 25 wt. % to 40 wt. % of the total amount of active agent in the dosage form is released in the initial pulse, and approximately 25 wt. % to 40 wt. % of the total amount of active agent in the dosage form is released in each of the second and third pulses. For dosage forms that mimic a three times daily dosing profile, release of the second pulse preferably takes place approximately 3 hours to 10 hours, and more preferably approximately 4 to 9 hours, following oral administration. Release of the third pulse occurs about 2 hours to about 8 hours following the second pulse, which is typically about 5 hours to approximately 18 hours following oral administration.

The dosage form can be a closed capsule housing at least two drug-containing dosage units, each dosage unit comprising one or more compressed tablets, or may be comprised of a plurality of beads, granules or particles, providing that each dosage unit has a different drug release profile. The immediate release dosage unit releases drug substantially immediately following oral administration to provide an initial dose. The delayed release dosage unit releases drug approximately 3 hours to 14 hours following oral administration to provide a second dose. Finally, an optional second delayed release dosage unit release drug about 2 hours to 8 hours following the release of the second dose, which is typically 5 hours to 18 hours following oral administration.

Another dosage form comprises a compressed tablet or a capsule having a drug-containing immediate release dosage unit, a delayed release dosage unit and an optional second delayed release dosage unit. In this dosage form, the immediate release dosage unit comprises a plurality of beads, granules or particles that release drug substantially immediately following oral administration to provide an initial dose. The delayed release dosage unit comprises a plurality of coated beads or granules, which release drug approximately 3 hours to 14 hours following oral administration to provide a second dose.

An optional second delayed release dosage unit comprises coated beads or granules that release drug about 2 to 8 hours following administration of the initial delayed release dose, which is typically 5 to 18 hours following oral administration. The breads or granules in the delayed release dosage unit(s) are coated with a bioerodible polymeric material. This coating prevents the drug from being released until the appropriate time, i.e., approximately 3 hours to less than 14 hours following oral administration for the delayed release dosage unit and at least 5 hours to approximately 18 hours following oral administration for the optional second delayed release dosage unit. In this dosage form the components may be admixed in the tablet or may be layered to form a laminated tablet.

Another dosage form is a tablet having a drug-containing immediate release dosage unit, a delayed release dosage unit, and an optional second delayed release dosage unit, wherein the immediate release dosage unit comprises an outer layer that releases the drug substantially immediately following oral administration. The arrangement of the remaining delayed release dosage(s), however, depends upon whether the dosage form is designed to mimic twice daily dosing or three times daily dosing.

In the dosage form mimicking twice daily dosing, the delayed release dosage unit comprises an inner core that is coated with a bioerodible polymeric material. The coating is applied such that release of the drug occurs approximately 3 hours to less than 14 hours following oral administration. In this form, the outer layer completely surrounds the inner core.

In the dosage form mimicking three times a day dosing, the (first) delayed release dose comprises an internal layer that releases drug approximately 3 hours to less than 14 hours following oral administration. This internal layer is surrounded by the outer layer. The second delayed release dosage unit generally comprises an inner core that releases the drug at least 5 hours to approximately 18 hours following oral administration. Thus, the layers of this tablet (starting from the external surface) comprise an outer layer, an internal layer and an inner core. The inner core comprises delayed release beads or granules. Furthermore, the internal layer comprises the drug coated with a bioerodible polymeric material. Alternatively, in this particular dosage form mimicking three times a day dosing, both the delayed release dosage unit and second delayed release dosage units are surrounded by an inner layer. This inner layer is free of active agent. Thus, the layers of this tablet (starting from the external surface) comprise an outer layer, inner layer and an admixture of the delayed release dosage units. The first delayed release pulse occurs once the inner layer is substantially eroded thereby releasing the admixture of the delayed release dosage units. The dose corresponding to the (first) delayed release dosage unit is released immediately since the inner layer has prevented access to this dose for the appropriate time, e.g., from approximately 3 hours to 10 hours. The second delayed release dose, however, is formulated to effectively delay release for at least 5 hours to approximately 18 hours following oral administration.

For formulations mimicking twice daily dosing, it is preferred that the delayed release dose is released approximately 3 hours to up to 14 hours, more preferably approximately 5 hours to up to 12 hours, following oral administration. For formulations mimicking three times daily dosing, it is preferred that the (first) delayed release dose is released approximately 3 to 10 hours, preferably 4 hours to 9 hours, following oral administration. For dosage forms containing a third dose, the third dose (e.g., the second delayed release dose) is released at least 5 hours to approximately 18 hours following oral administration.

In still another embodiment, a dosage form is provided which comprises a coated core-type delivery system wherein the outer layer is comprised of an immediate release dosage unit containing an active agent, such that the active agent therein is immediately released following oral administration; an intermediate layer there under which surrounds a core; and a core which is comprised of immediate release beads or granules and delayed release beads or granules, such that the second dose is provided by the immediate release beads or granules and the third dose is provided by the delayed release bead or granules.

Drug complexes are generally prepared by complexing the drug with a pharmaceutically acceptable ion-exchange resin. The complex is formed by reaction of a functional group of the drug with a functional group on the ion exchange resin. Drug is released by exchanging with appropriately charged ions within the gastrointestinal tract.

Ion-Exchange Resins

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups. The covalently bound salt forming groups may be strongly acidic (e.g., sulfonic acid or sulfuric acid) or weakly acidic (e.g., carboxylic acid). In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975).

Resins include Amberlite IRP.®.-69 (Rohm and Haas) INDION 224, INDION 244, and INDION 254 (Ion Exchange (India) Ltd.). These resins are sulfonated polymers composed of polystyrene cross-linked with divinylbenzene. Any ion-exchange resins currently available and those that should become pharmaceutically acceptable and available in the future can also be used. Commercial sources of ion exchange resins that are either pharmaceutically acceptable or may become pharmaceutically acceptable in the future include, but are not limited to, Rohm and Haas, The Dow Chemical Company, and Ion Exchange (India) Ltd.

The size of the ion-exchange particles should be less than about 2 millimeter, more preferably below about 1000 micron, more preferably below about 500 micron, and most preferably below about 150 micron. Commercially available ion-exchange resins (Amberlite IRP.®.-69, INDION.®. 244 and INDION.®. 254) have a particle size range less than 150 microns.

Drug is bound to the resin by exposure of the resin to the drug in solution via a batch or continuous process (such as in a chromatographic column). The drug-resin complex thus formed is collected by filtration and washed with an appropriate solvent to insure removal of any unbound drug or by-products. The complexes are usually air-dried in trays. Such processes are described in, for example, U.S. Pat. Nos. 4,221,778 to Raghunathan; 4,894,239 to Nonomura; and 4,996,047 to Kelleher.

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt form); and (d) resin (H-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

The resin-drug complexes can be incorporated into tablets, capsules, beads, films, coatings or particles. The resin-drug complexes or particles containing the complexes can also be suspended in a liquid such as an enteric coating or barrier to alter release properties. Complexes with different coatings, or mixture of uncoated with coated complexes or particles, can be used to create mixtures with different release properties.

### III. Methods of Manufacturing Dosage Forms

As will be appreciated by those skilled in the art and as described in the pertinent texts and literature, a number of methods are available for preparing drug-containing tablets, beads, capsules, granules or particles, films, solutions, suspensions, and coatings that provide a variety of drug release profiles. Such methods include, but are not limited to, coating a drug or drug-containing composition with an appropriate coating material, increasing drug particle size, placing the drug within a matrix of excipient and other fillers, coating the material with an enteric coating, and forming complexes of the drug with a suitable complexing agent such as an ion-exchange resin. In one embodiment, the pharmaceutical composition is in the form of orally disintegrating immediate release tablet. In another embodiment, the pharmaceutical composition is in the form of orally disintegrating controlled release tablet. An orally disintegrating tablet (ODT) is a single unit dose that disintegrates in the oral cavity.

Coatings can be aqueous or organic. Film coatings are typically thin barrier films, providing protection or color to the particles or tablets. Active ingredient can be incorporated into the coating. Coatings may be formed of lipids or by hot melting of polymers. This provides coatings of between 25 and several hundred microns in thickness. These coatings protect against moisture. No evaporation of solvents is required. Sugar coatings are generally between 0.5 and 2 mm thick. These are used to provide taste masking and sealing, as well as for protection and coating of temperature-sensitive and fragile products. The coating is applied by spraying of a syrup onto the particles.

These sprayed coatings can vary between approximately 5 microns and 50 microns or more in thickness. Coatings can be applied as polymeric solutions or sprays by fluidized bed reactors, by spray coating (top spray, Worster coating—bottom spray), or tangential spray—rotor pellet coating), or drum or pan coaters. Top spray coatings are used for general coatings including enteric coatings. Top spray coatings are used for general coatings including enteric coatings. Particles are fluidized in the flow of heated air, which is introduced into the product container, then the coating liquid is sprayed into the fluid bed from above. Drying takes place as the particles move upward. Bottom spraying is particularly suitable for controlled release of active ingredients. In the Wurster process, a complete sealing of the surface can be achieved with a low usage of coating substance. The spray nozzle is fitted in the base plate resulting in a spray pattern that is concurrent with the air feed. By using a Wurster cylinder and a base plate with different perforations, the particles to be coated are accelerated inside the Wurster tube and fed through the spray cone concurrently. As the particles continue traveling upwards, they dry and fall outside back to the inside of the tube where they are once again accelerated by the spray. This produces an extremely even film. Particles of different sizes are evenly coated. Particularly suitable for protective coatings/color coatings where the product throughput rates are high. For continuous fluid bed coatings, the product is continuously fed into one side of the machine and is transported onwards via the sieve bottom by means of the air flow. Depending on the application, the system is sub-divided into pre-heating zones, spray zones and drying zones whereby spraying can take place from below in the form of a bottom spray. The dry, coated particles are continuously extracted. Tangential spray coatings (Rotor pellet coating) are ideal for coatings with high solid content. The product is set into a spiral motion by means of a rotating base plate, which has air fed into the powder bed at its edge. The spray nozzle is arranged tangentially to the rotor disc and also sprays concurrently into the powder bed. Very thick film layers can be applied by means of the rotor method. Tablets and dragees are coated using drum or pan coates. These are typically for the application of protective films or taste masking.

Powder particles can be agglomerated in a fluid bed to build up powder granulates, typically in the size range of 0.2 and 2.5 mm. The powder is moistened in order to form liquid bridges between the particles. The spray liquid can be either water or an organic solvent which dissolves the powder or a binder. The moistened granulates are dried and cooled. The granulates have a low bulk density and are highly water soluble. Wet granulation is used to build up granulates from powder. Wet granulates are generally denser and more mechanically stable particles than fluid bed granulates and produce grains between 0.1 and 10 mm. Wet granulation in a vertical granulator is the classical method for building up granulates from powder. In this process, powder is fed to a product container and then moistened or sprayed with molten material in order to increase the cohesive forces. The liquid can be water or an organic solvent, optionally containing a binder. The ingredients are simultaneously mixed together vigorously. Denser granulates are formed than in the case of the fluid bed process. The products are highly suitable for making into tablets, which are compact and exhibit low hygroscopicity. Spray granulation is the drying of liquids (solutions, suspension, melts) while simultaneously building up granulates.

If the matrix material is dissolved in the liquid phase, the granulates are made by means of spray granulation. If the matrix material is present in the form of powder, the granulates are made by means of wet granulation. This encapsulation process is mainly applied in the food industry. If necessary, the sprayed granulates can be provided with a protective coating in an additional step.

Blending is the dry mixing of ingredients to produce a uniform distribution of components. In solid processes, various individual products of different density and concentration and in different amounts are often admixed to form a homogeneous mixture. In the pharmaceutical area, very different quantities and proportions of active and auxiliary ingredients (corn starch, lactose, PVP, etc.) are mixed together. Specific auxiliary materials such as lubricants or flavorings may also be added. Mixing may be necessary in different process sections. For instance, compression aids, flow controlling media and external phases are added following the granulation process and before compression.

Direct pelletizing is the manufacture of pellets directly from powder. Pelletizing can occur by building up layer by layer around a starting core, or a round pellet can be extruded by spheronizing. Spray granulation can also be used for the build-up of liquid particles. In direct pelletizing, pellets are manufacture directly from powder with a binder or solvent. This is a fast process and yields compact, round pellets, which have a higher density than spray granulates and agglomerates. Pellet diameters are between 0.2 and 1.2 mm. Pellets can be made into tablets are used to fill capsules. Pelletizing by layering results in build-up layer by layer of material around a given starting core. This is ideal for forming round pellets with separate layers of powder coatings and/or active agent. The layers are densely applied due to the movement of the pellets in the rotor. Thick layers can be applied to the starting grains, which allow large amounts of active to be incorporated. These have a higher density than spray granulates and agglomerates. Typical diameters are between 0.6 and 2.5 mm. In spheronizing, round pellets are formed from irregular wet granulates and extruded products. The moist granulates or extruded products are fed onto a rotating/palletizing plate. The surface is smoothed due to the intensive rolling movement and spherical pellets are produced due to the intensive rolling movement. This results in narrow particle size distribution and good flow behavior. Pellets have a higher density than spray granulates and agglomerates. Typical particle diameters are between 0.5 and 2.5 mm. Spray granulation is the drying of liquids (solutions, suspensions, melts) while simultaneously building up of granulates. These are denser and harder than agglomerates and have a size between 0.2 and 5 mm.

For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6th Ed. (Media, Pa.: Williams & Wilkins 1995)). A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidione, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### IV. Methods of Administration

The amount of pharmaceutical agent(s) and the amount of immunonutritional agent, and the type (time and rate) of release of each agent from the formulations following administration to a patient may vary depending upon multiple factors including, but not limited to, the particular pharmaceutical agent or immunonutritional agent, the patient's degree of illness, the patient's weight, and the patient's age.

In a preferred embodiment, the pharmaceutical formulations are used to treat and/or prevent rhinitis. The pharmaceutical formulations and immunonutritional compositions are typically administered orally. The pharmaceutical formulations and/or immunonutritional compositions may also be formulated for buccal administration or sublingual administration. Other methods of administration include nasal or oral sprays, or through the use of transdermal delivery vehicles, such as adherent transdermal patches.

The kit provides multiple dosing units of each agent in a convenient, well-marked container, such as a blister card or bottle, together with instructions as to the dosing and use of each agent. For liquid formulations of the immunonutritional composition and/or the pharmaceutical composition, the kit can contain a unit dose liquid packaging. Examples of unit dose liquid packaging include, but are not limited to, flow fill seal dosing units. The concept of aseptic blow fill seal (BFS) is that a container is formed, filled, and sealed in a continuous process without human intervention in a sterile enclosed area inside a machine. The process is multi-stepped, pharmaceutical grade resin is extruded into a tube, which is then formed into a container. A mandrel is inserted into the newly formed container and filled. The container is then sealed, all inside a sterile shrouded chamber inside the machine. The product is then discharged to a non-sterile area for packaging and distribution.

To use the kit, a patient reads the instructions, and orally administers the one or more pharmaceutical formulations and the one or more immunonutritional compositions at the prescribed times. The administration of the immunonutritional composition in combination with the pharmaceutical formulations is designed to reduce the severity and or duration of infectious rhinitis, as compared to when the same pharmaceutical formulations are administered in the absence of the immunonutritional composition.

In one embodiment, the kit contains acetate lozenges and an oral solution containing guaifenesin, carbetapentane citrate, and phenylephrine hydrochloride. In another embodiment, the kit contains zinc acetate lozenges and an oral solution containing hydrocodone bitartrate, guaifenesin, and phenylephrine hydrochloride. In still another embodiment, the kit contains zinc acetate lozenges and chewable tablets containing chlorpheniramine maleate, phenylephrine hydrochloride, and methscopolamine nitrate. In yet another embodiment, the kit contains zinc acetate lozenges and tablets containing pseudoephedrine hydrochloride and chlorpheniramine maleate.

Modifications and variations will be apparent to those skilled in the art and are intended to be encompassed by the following claims. All publications cited herein are incorporated by reference.

### EXAMPLES

### Example 1

### A Kit Containing Zinc Acetate Lozenges and an Oral Solution Containing Guaifenesin, Cabetapentane Citrate, and Phenylephrine Hydrochloride

A kit containing zinc acetate lozenges and an oral solution containing guaifenesin, carbetapentane citrate, and phenylephrine hydrochloride was prepared with excipients within the disclosed ranges. The amounts of the components in the zinc lozenges are shown in Table 1. The amounts of the components in the oral solution are shown in Table 2.

TABLE 1
Amounts of the components in the zinc lozenge

| Active Component | Amount (mg) |
|---|---|
| Zinc acetate | Equivalent to 14 mg of zinc |

| Inactive Components | Amount (wt %) |
|---|---|
| Dextrose | 20-90 |
| Glyceryl monostearate | 0.01-10 |
| Colloidal silicon dioxide | 0.1-1.5 |
| Peppermint oil | 0.01-5 |
| Stevia | 0.01-10 |

TABLE 2
Concentrations of the components in the oral solution

| Active Components | | Concentration (mg/ml of solution) |
|---|---|---|
| Guaifenesin | | 20 |
| Carbetapentane citrate | | 3 |
| Phenylephrine hydrochloride | | 1 |
| | Inactive Components | Concentration (wt %) |
| | Methylparaben | 0.015-0.2 |
| | Propylparaben | 0.001-0.02 |
| | Propylene Glycol | 3-25 |
| | Potassium citrate | 0.3-2.0 |
| | Glycerin | 5-50 |
| | Maltitol | 2-65 |
| | Sorbitol | 2-70 |
| | Sodium saccharin | 0.01-1 |
| | Purified water | qs |

### Example 2

### A Kit Containing Zinc Lozenges and an Oral Solution Containing Hydrocodone Bitartrate, Guaifenesin, and Phenylephrine Hydrochloride

A kit containing zinc acetate lozenges and an oral solution containing hydrocodone bitartrate, guaifenesin, and phenylephrine hydrochloride was prepared with excipients within the disclosed ranges. The composition of the zinc lozenge is the same as described in Example 1. The amounts of the components in the oral solution are shown in Table 3.

TABLE 3
Concentrations of the components in the oral solution

| Active Components | Concentration (mg/ml of solution) |
|---|---|
| Hydrocodone bitartrate | 0.5 |
| Guaifenesin | 20 |
| Phenylephrine hydrochloride | 1 |

| Inactive Components | Amount (wt %) |
|---|---|
| Glycerin | 5-50 |
| Maltitol | 2-65 |
| Propylene glycol | 3-25 |
| Sorbitol | 5-70 |
| Sodium saccharin | 0.01-1 |
| Grape flavor | 0.001-5 |
| FD&C Red #40 | 0.001-5 |
| FD&C Blue #1 | 0.001-5 |
| Purified Water | qs |

### Example 3

### A Kit Containing Zinc Acetate Lozenges and Chewable Tables Containing Phenylephrine Hydrochloride, Chlorpheniramine Maleate, and Methscopolamine Nitrate

A kit containing zinc acetate lozenges and chewable tables containing phenylephrine hydrochloride, chlorpheniramine maleate, and methscopolamine nitrate was prepared with excipients within the disclosed ranges. The composition of the zinc lozenge is the same as described in Example 1. The amounts of the components in the chewable tablet are shown in Table 4.

TABLE 4
Concentrations of the components in the chewable tablet

| Active Components | Amount (mg/tablet) |
|---|---|
| Phenylephrine hydrochloride | 10 |
| Chlorpheniramine maleate | 2 |
| Methscopolamine nitrate | 1.25 |

| Inactive Components | Amount (wt %) |
|---|---|
| Iron oxide | 0.001-5 |
| Dye | 0.001-5 |
| Mannitol | 5-90 |
| Magnesium stearate | 0.01-3 |
| Sugar | 5-50 |
| Microcrystalline cellulose | 2-50 |
| Root beer flavor | 0.001-5 |

### Example 4

### A Kit Containing Zinc Acetate Lozenges and an Oral Suspension Containing Carbetapentane Tannate and Phenylephrine Tannate

A kit containing zinc acetate lozenges and an oral suspension containing carbetapentane tannate and phenylephrine tannate was prepared with excipients within the disclosed ranges. The composition of the zinc lozenge is the same as described in Example 1. The amounts of the components in the oral suspension are shown in Table 5.

TABLE 5
Concentrations of the components in the oral suspension

| Active Components | Concentration (mg/mL) |
|---|---|
| Carbetapentane tannate | 6 |
| Phenylephrine tannate | 5 |

| Inactive Components | Amount (wt %) |
|---|---|
| Potassium sorbate | 0.005-0.2 |
| Methylparaben | 0.015-0.2 |
| Propylparaben | 0.001-0.02 |
| Propylene glycol | 3-25 |
| Glycerin | 5-50 |
| Potassium citrate | 0.3-2 |
| Xanthan gum | 0.01-10 |
| Citric acid | 0.1-2.0 |
| Aspartame | 0.001-10 |
| Flavoring agent | 0.001-5 |
| Dye | 0.001-5 |
| Purified water | qs |

### Example 5

### A Kit Containing Zinc Acetate Lozenges and Capsules Containing Pseudoephedrine Hydrochloride and Chlorpheniramine Maleate

A kit containing zinc acetate lozenges and capsules containing pseudoephedrine hydrochloride and chlorpheniramine maleate was prepared with excipients within the disclosed ranges. The composition of the zinc lozenge is the same as described in Example 1. The amounts of the components in the capsules are shown in Table 6.

TABLE 6
Concentrations of the components in the capsules

| Active Components | Amount (mg/tablet) |
|---|---|
| Pseudoephedrine hydrochloride | 120 |
| Chlorpheniramine maleate | 8 |

| Inactive Components | Amount (wt %) |
|---|---|
| Shellac | 5-35 |
| Lactose | 5-75 |
| Microcrystalline cellulose | 5-75 |
| Ethylcellulose | 1-20 |
| Hydroxypropylcellulose | 5-35 |
| Hypromellose | 2-20 |
| Triacetin | 10-35 |

What is claimed is:

1. A kit for treating rhinitis, comprising at least one pharmaceutical formulation comprising a pharmaceutical agent effective for the treatment of symptoms associated with rhinitis and at least one immunonutritional composition.

2. The kit of claim 1, wherein the immunonutritional composition comprises a pharmaceutically acceptable form of zinc.

3. The kit of claim 1, wherein the immunonutritional composition further comprises vitamins, additional minerals and flavorings.

4. The kit of claim 1, wherein the pharmaceutical agent is selected from the group consisting of antihistamines, decongestants, anticholinergics, antitussives, analgesics, antipyretics, mucolytics, expectorants, and combinations thereof.

5. The kit of claim 1, wherein the pharmaceutical formulation further comprises a second pharmaceutical agent.

6. The kit of claim 1, further comprising instructions containing a dosing schedule.

7. The kit of claim 1, wherein the kit comprises labeling distinguishing the pharmaceutical formulation from the immunonutritional composition.

8. The kit of claim 1, further comprising one or more packages selected from the group consisting of bottles, blister packs, blow fill seal packages, and combinations thereof.

9. The kit of claim 8, wherein the pharmaceutical formulation is in the form of a liquid.

10. The kit of claim 8, wherein the immunonutritional composition is in the form of a liquid.

11. The kit of claim 9, wherein the pharmaceutical composition is packaged in a liquid unit dosage form.

12. The kit of claim 11, wherein the liquid unit dosage form is a blow fill seal dosage form.

13. The kit of claim 10, wherein the immunonutritional composition is packaged in a liquid unit dosage form.

14. The kit of claim 13, wherein the liquid unit dosage form is a blow fill seal dosage form.

15. The kit of claim 1, wherein the pharmaceutical formulation is in a dosage form suitable for providing controlled release of the pharmaceutical agent.

16. The kit of claim 15, wherein the controlled release comprises immediate, sustained, modified, delayed, pulsed release, or combinations thereof.

17. The kit of claim 1, wherein the pharmaceutical formulation is in a dosage form suitable for providing immediate release of the pharmaceutical agent.

18. The kit of claim 1, wherein the immunonutritional composition is in the form of a solid.

19. The kit of claim 18, wherein the immunonutritional composition is in the form of a lozenge.

20. The kit of claim 1, further comprising a blister pack.

21. The kit of claim 2, wherein the pharmaceutical formulation is in the form of a nasal spray.

22. The kit of claim 1, wherein the pharmaceutical formulation is in the form of a gel.

23. The kit of claim 1, wherein the immunonutritional formulation is in the form of a nasal spray.

24. The kit of claim 1, wherein the immunonutritional formulation is in the form of a gel.

25. The kit of claim 1, wherein the pharmaceutical formulation is in the form of a film for lingual or buccal administration.

26. The kit of claim 1, wherein the pharmaceutical formulation is in the form of an orally disintegrating immediate release tablet.

27. The kit of claim 1, wherein the pharmaceutical formulation is in the form of an orally disintegrating controlled release tablet.

28. A method of treating rhinitis comprising administering to a patient in need thereof a kit comprising at least one pharmaceutical formulation comprising a pharmaceutical agent effective for the treatment of symptoms associated with rhinitis and at least one immunonutritional composition.

29. The method of claim 28, wherein the immunonutritional composition comprises a pharmaceutically acceptable form of zinc.

30. The method of claim 28, wherein the immunonutritional composition further comprises vitamins, additional minerals and flavorings.

31. The method of claim 28, wherein the pharmaceutical agent is selected from the group consisting of antihistamines, decongestants, anticholinergics, antitussives, analgesics, antipyretics, mucolytics, expectorants, and combinations thereof.

32. The method of claim 28, wherein the pharmaceutical formulation further comprises a second pharmaceutical agent.

33. The method of claim 28, further comprising instructions containing a dosing schedule.

34. The method of claim 28, wherein the kit comprises labeling distinguishing the pharmaceutical formulation from the immunonutritional composition.

35. The method of claim 28, further comprising one or more packages selected from the group consisting of bottles, blister packs, blow fill seal packages, and combinations thereof.

36. The method of claim 35, wherein the pharmaceutical formulation is in the form of a liquid.

37. The method of claim 35, wherein the immunonutritional composition is in the form of a liquid.

38. The method of claim 28, wherein the pharmaceutical formulation is in a dosage form suitable for providing controlled release of the pharmaceutical agent.

39. The method of claim 38, wherein the controlled release comprises immediate, sustained, modified, delayed, pulsed release, or combinations thereof.

40. The method of claim 28, wherein the pharmaceutical formulation is in a dosage form suitable for providing immediate release of the pharmaceutical agent.

41. The method of claim 28, wherein the immunonutritional composition is in the form of a solid.

42. The method of claim 38, wherein the immunonutritional composition is in the form of a lozenge.

43. The method of claim 28, further comprising a blister pack.
